(19) United States
(12) Patent Application Publication
(10) Pub. No.: US 2013/0089603 A1
(43) Pub. Date: Apr. 11, 2013

(54) COMPOSITIONS AND METHODS FOR TREATING OBESITY AND OBESITY-RELATED CONDITIONS

(71) Applicant: Ronald Thompson, Fort Thomas, KY (US)

(72) Inventor: Ronald Thompson, Fort Thomas, KY (US)

(73) Assignee: CHELATEXX LLC, Fort Thomas, KY (US)

(21) Appl. No.: 13/577,848

(22) PCT Filed: Jul. 22, 2010
(86) PCT No.: PCT/US10/42835

§ 371 Date: Sep. 20, 2012

Related U.S. Application Data
(63) Continuation-in-part of application No. 12/658,331, filed on Feb. 8, 2010, now Pat. No. 8,110,186.
Application No. 12/658,331 is a continuation-in-part of application No. 11/654,361, filed on Jan. 16, 2007, now Pat. No. 7,662,373.
Application No. 11/654,361 is a division of application No. 11/522,627, filed on Sep. 18, 2006, now abandoned.

(51) Int. Cl. A61K 33/44; A61K 9/48; A61K 31/337

(52) U.S. Cl. CPC A61K 33/44; A61K 31/337; A61K 9/4808
USPC 424/451; 424/125; 424/400; 424/464

(57) ABSTRACT

A method to effectively treat the adverse events of ingested lipase inhibitor such as orlistat, and to maintain the effectiveness of ingested orlistat, the method comprising the steps of: ingesting a compound of orlistat to irreversibly bind with lipase enzymes of the gastrointestinal tract; ingesting a compound of simethicone to cause undigested fats to remain in an emulsified state in the bowel; and ingesting an enteric-coated activated charcoal to absorb emulsified fats only in the lower bowel, thus preventing the adverse events associated with the ingestion of orlistat alone.

This application is a continuation-in-part of U.S. patent application Ser. No. 12/658,331, filed Feb. 8, 2010, entitled “Method to Maintain the Efficacy of Orlistat,” which is a continuation-in-part of U.S. Pat. No. 7,662,373, issued Feb. 16, 2010, entitled “Method and Composition of a Medicament to Decrease the Adverse Events of Olistat, an Oral Lipase Inhibitor,” which is a divisional of U.S. patent application Ser. No. 11/522,627, filed Sep. 18, 2006, entitled “Method and Composition of a Medicament to Decrease the Adverse Events of Olistat, an Oral Lipase Inhibitor.” Applicant claims the benefit of each of the foregoing patents and patent applications. In addition, the disclosures of each of the foregoing patents and patent applications are incorporated by reference herein.

### BACKGROUND

Current medical literature has documented over 400 U.S. deaths directly caused by weight loss drugs. These deaths have been attributed to hemorrhagic strokes, cardiac arrests, and even suicide. The weight loss drugs in question were anorectics, which not only reduce appetite but are also central nervous system (CNS) stimulants. Recently, the U.S. Food and Drug Administration (FDA) has mandated that pharmaceutical companies remove numerous weight loss drugs from the market because of deaths and serious complications caused by anorectic weight loss products. All of these CNS stimulating anorectic drugs were FDA-approved and indicated for physician monitored weight loss. For example, in 1996 it was discovered that the most widely prescribed weight loss drugs (FEN-PHEN/REDUX) had been causing two life threatening conditions: cardiac valve damage and primary pulmonary hypertension. As a result, the pharmaceutical industry has paid billions of dollars due to class-action lawsuits. FEN-PHEN/REDUX, including their generics such as fenfluamine and dexfenfluamine, are proven weight loss drugs classified as anorectics. These particular sympathomimetic amines acting as CNS stimulants have been shown to significantly increase the risk of cardiac valve damage and primary pulmonary hypertension if used over an extended period of time.

Recently, various non-anorectic weight loss drugs have been developed and introduced into the market. One such type of product is lipase inhibitors. Pancreatic lipase is an enzyme that breaks down digested fats into small chain fatty acids in the lower lumen of the stomach, and in the small intestine. Various compounds which inhibit the activity of pancreatic lipase have been developed. By inhibiting the actions of the pancreatic lipase enzyme, these lipase inhibitors reduce the absorption of ingested fats and are therefore used for weight loss. One such lipase inhibitor is orlistat (also known as tetrahydrolipstatin). Orlistat is marketed under the trade names Xenical® (Roche) and alli® (GlaxoSmithKline). In the U.S., Xenical is a prescription drug product containing 120 mg of orlistat per unit dose, whereas Alli is sold over-the-counter and contains 60 mg of orlistat per unit dose.

Orlistat (tetrahydrolipstatin) and related lipase inhibitors are described in U.S. Pat. No. 4,598,089, issued Jul. 1, 1986, entitled “Leucine Derivatives.” These actions are further defined in U.S. Pat. Nos. 5,245,056 and 5,399,720, to treat obesity and various medical conditions associated with obesity. U.S. Pat. No. 6,696,467 further teaches and defines the specific benefits of the lipase inhibitor tetrahydrolipstatin (orlistat) for the treatment of obesity by weight reduction and appetite suppression. A pharmaceutical composition containing orlistat, as well as methods for preparing such compositions, is described, for example, in U.S. Pat. No. 6,004,996, issued Dec. 21, 1999, entitled “Tetrahydrolipstatin Containing Compositions.” This patent describes the production of tetrahydrolipstatin into microspheres for the optimal therapeutic delivery into the lumen of the stomach. These microspheres have very efficient action as a lipase inhibitor because of the large surface area to bind to the lipase inhibitor. U.S. Pat. No. 6,607,749, issued Aug. 19, 2003, entitled “Lipstatin Derivative-Soluble Fiber Tablets,” describes a dual therapy to treat adiposity with orlistat and to treat the fecal incontinence associated with orlistat, concurrently. Another lipase inhibitor is cetilistat. Cetilistat and related lipase inhibitors are described in U.S. Pat. No. 7,407,954, issued Aug. 5, 2008, entitled “Thieno-(1,3)-oxazin-4-ones with Lipase Inihibiting Activity.” The disclosure of each of the above-cited patents is incorporated by reference herein.

Unlike the anorectics, orlistat and other lipase inhibitors are not systematically absorbed and act only in the bowel. Orlistat does not stimulate neurons, is not a neurotransmitter, is not a sympathomimetic amine, and is not an anorectic agent. Orlistat is a lipase inhibitor, and generates weight loss by preventing the digestion and absorption of 25-35% of ingested fat. Orlistat, a non-systemically absorbed lipase inhibitor, was initially FDA-approved as safe and effective in 1999. Further, orlistat's safety was re-affirmed when the FDA allowed it to be converted from prescription status to over-the-counter (with the dosage reduced from 120 mg to 60 mg). OTC status confers such a high degree of confidence that a product is safe that a physicians oversight is not even required.

While orlistat and other lipase inhibitors are effective for treating obesity by reducing the absorption of ingested fats, there are several gastrointestinal adverse events (i.e., side effects) associated with the use of these drugs. These adverse events generally result from the passage of undigested fats through the gastrointestinal tract. The Physicians Desk Reference lists the adverse events of orlistat clinical trials on over 2800 patients for one or two years as:

Upper gastrointestinal adverse events:

| | |
|---|---|
| Abdominal pain/discomfort | 25.5% |
| Nausea | 8.1% |

Lower gastrointestinal adverse events:

| | |
|---|---|
| Oily Spotting | 26.6% |
| Flatus - with discharge | 23.9% |
| Fecal urgency | 22.1% |
| Fatty/Oily stool | 26.0% |
| Oily Evacuation | 11.9% |
| Increased defecation | 10.8% |
| Fecal incontinence | 7.7% |

Orlistat, as a lipase inhibitor, creates a mal-absorption state in which ingested fats are not absorbed in the small intestine, and therefore must be eliminated through the lower intestines and rectum. All of the adverse events are directly caused by the elimination of large sized fat globules.

Table 7.3 of the FDA published “Orlistat Advisory Committee Briefing Document,” published in the Federal Register on Jan. 13, 2007, documented that 60 mg of orlistat caused adverse events in 89.1% of study participants, and that 120 mg of orlistat caused adverse events in 91.4% of the study participants. The “Orlistat Advisory Committee Briefing Document” also reported that the 60 mg dose of orlistat (OTC) will prevent the digestion and absorption of 25% of ingested fat, while the 120 mg dose of orlistat (prescription) will prevent the digestion and absorption of 30% ingested fat. The undigested and unabsorbed ingested fats are the etiology of all the adverse events of orlistat use.

While orlistat has been shown to be effective in promoting weight loss, the adverse events associated with its use have resulted in lower than projected sales. Even the reduced OTC dosage has not eliminated these adverse events. Actual over-the-counter orlistat sales have only been a fraction of the projected sales because of the lack of product acceptance by the public. The lack of product acceptance is because of the socially unacceptable orlistat adverse events of flatus with discharge and involuntary oily rectal discharge. The orlistat adverse events are reported by over 50% of individuals even on a rigid low fat diet, and over 90% of individuals on a normal (25 grams of fat/meal) US diet, when using orlistat for weight loss.

In an effort to reduce the adverse events associated with the use of orlistat, the marketer of OTC orlistat instructed consumers that they must commit to a low fat diet of no more than 15 grams/meal of fat and be prepared for the orlistat-induced adverse events. The latter included instructing consumers to, for example: “Wear dark pants to work when you use alli™”; “Take an extra pair of underwear to work when you are using alli™”; and “Start alli™ on a weekend when you can be home.”

The marketers of orlistat also attempted to develop a way to eliminate or reduce the adverse events associated with orlistat. Despite a 10-year research and development project initiated by Hoffman LaRoche, and continued by GlaxoSmithKline, to develop an antidote or controlling agent to prevent the orlistat adverse events of flatus with discharge and involuntary rectal spotting, these efforts have been unsuccessful. Thus, those skilled in the art recognize the failures of everyone to date in overcoming the “adverse effects” of orlistat. The industry is still searching for a solution to the orlistat “treatment effects” while maintaining the efficacy of the orlistat treatment. In fact, as of the filing date of this application, GlaxoSmithKline's own website still states that they are looking for an “[i]ngredient or formulation technology to bind the loose fat in the bowels without affecting efficacy of Orlistat.”

While the marketer of orlistat has defined the problem as steatorrhea, this is not accurate. The real problem, and the reason for less-than-anticipated acceptance of orlistat by consumers, is “underwear issues.” Steatorrhea is defined as fatty stools. Steatorrhea is a reported sign of mal-absorption and defines a differential diagnosis of the etiology of the steatorrhea. However, individuals experiencing steatorrhea do not experience flatus with discharge or oily spotting. Orlistat induces a mal-absorption state and therefore will induce steatorrhea, but to convince individuals to maintain a very low fat diet for an extended period of time will meet with limited success. One does not typically become overweight by eating a low fat diet, and therefore most people will not commit to maintain a low fat diet just to use orlistat to induce weight loss. Once again, the steatorrhea does not cause the “underwear issues” of flatus with discharge and oily spotting.

While a variety of compositions and methods may exist for treating obesity and obesity-related disorders, it is believed that no one prior to the inventor has made or used an invention as described herein.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings. In the drawings, like numerals represent like elements throughout the several views.

FIG. 1 is a diagrammatic representation of a human gastrointestinal tract, labeled to indicate significant portions thereof.

FIG. 2 is a view similar to FIG. 1, about one-half of an hour after a meal has been consumed along with orlistat, simethicone and enteric-coated activated charcoal.

FIG. 3 is a view similar to FIG. 2, about one hour after the meal has been consumed.

FIG. 4 is a view similar to FIG. 3 about two hours after the meal has been consumed.

FIG. 5 is a partial cross-sectional view of an exemplary capsule containing orlistat, simethicone and enteric-coated activated charcoal.

FIG. 6 is a schematic top plan view of a blister package of three unit doses of the capsule of FIG. 5.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples should not be used to limit the scope of the present invention. Other features, aspects, and advantages of the versions disclosed herein will become apparent to those skilled in the art from the following description, which is by way of illustration, one of the best modes contemplated for carrying out the invention. As will be realized, the versions described herein are capable of other different and obvious aspects, all without departing from the invention. Accordingly, the drawings and descriptions should be regarded as illustrative in nature and not restrictive.

The present description provides compositions (or medicaments) for treating obesity or an obesity-related condition in a mammalian subject. Methods for treating obesity or an obesity-related condition are also provided, as well as methods for decreasing or preventing the adverse events associated with the use of lipase inhibitors (e.g., orlistat or cetilistat). Methods for decreasing or preventing the adverse events associated with the use of lipase inhibitors (e.g., orlistat or cetilistat) while maintaining the efficacy of the lipase inhibitor are also provided. Commercial packages containing one or more of the compositions or medicaments described herein are also provided. The term “obesity-related condition” refers to any disease or condition that is caused by or associated with (e.g., by biochemical or molecular association) obesity or that is caused by or associated with weight gain and/or related biological processes that precede clinical obesity. Examples of obesity-related conditions include, but are not limited to, metabolic syndrome, diabetes (e.g., type 1 diabetes, type 2 diabetes, and gestational diabetes), hyperlipidemia, hypertension, and dyslipidemia.

The Underwear Issues (UI) associated with the use of orlistat and other lipase inhibitors (e.g. cetilistat), namely flatus with discharge and oily spotting, can be controlled and even prevented by using a combination of a surfactant (or emulsifier) such as simethicone and fat-absorbing activated charcoal in conjunction with the lipase inhibitor. The surfactant acts in the upper GI tract and ensures that undigested fats remain in a non-globular state so that an oil slick is not presented to the sensory proprioceptors in the sigmoid colon. In addition, the surfactant ensures that the undigested fats are in a small enough droplet state to be absorbed by the activated charcoal. The activated charcoal (also referred to as activated carbon) absorbs the emulsified, undigested fats, thus minimizing or preventing the Underwear Issues associated with the use of orlistat and other lipase inhibitors. In some examples, the activated charcoal is provided in an enteric coated form. The enteric coating will not only maintain the efficacy of the lipase inhibitor, it also minimizes the absorption of nutrients by the charcoal. By minimizing or preventing the Underwear Issues related to orlistat use with a moderate fat diet, orlistat can gain better product acceptance. Increased orlistat product acceptance will allow many more individuals to achieve significant long term weight loss.

Before discussing in detail exemplary compositions and methods for, among other things, reducing the adverse events associated with the use of orlistat or other lipase inhibitors, it is useful to first discuss the GI tract and the medical model for the Underwear Issues associated with orlistat and other lipase inhibitors.

Gastrointestinal Tract

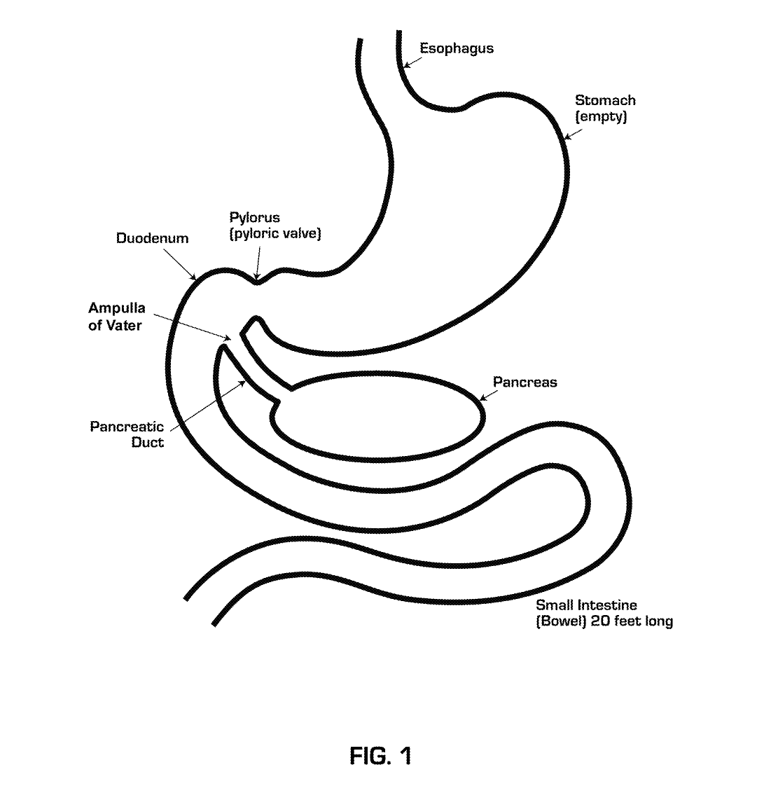

The gastrointestinal (GI) tract is divided into three major regions: (1) Upper GI—mouth, esophagus, stomach; (2) Small Intestines—20 ft long, referred to as the ‘bowel’ by surgeons; and (3)Large Intestines—10 ft long, referred to as the ‘colon’ by surgeons. FIG. 1 schematically depicts a portion of the human GI tract beginning uppermost, with the lower portion of the esophagus leading to the stomach, shown empty. The stomach leads to the pylorus (the pyloric valve) and the duodenum. The duodenum is subsequently divided at the ampulla of Vater into the pancreatic duct connecting the pancreas to the small intestine. The large intestine is not depicted in FIG. 1.

In response to consuming a meal, the stomach becomes distended and the pancreas secretes pancreatic lipase enzyme into the pancreatic duct. The pancreatic lipase enzyme travels through the ampulla of Vater into the duodenum (the initial segment of the bowel). The stomach rhythmically expels its contents into the duodenum where the food is admixed with the pancreatic lipase enzyme. The pyloric valve prevents any backflow reflux. The enzyme breaks down the ingested fats so that they may be absorbed in the small intestine. If orlistat (or other lipase inhibitor) is ingested with the meal, the orlistat binds with and inactivates the pancreatic lipase enzyme in the duodenum. The orlistat forms an irreversible bond with the pancreatic lipase enzyme and prevents the bound enzyme from breaking down ingested fats. This results in reduced absorption of fats, leading to weight loss. By way of example, a 60 mg does of orlistat has been shown to prevent the digestion and absorption of 25% of ingested fat (by fecal fat analysis). And a 120 mg does of orlistat has been shown to prevent the digestion and absorption of 30% of ingested fat. However, the unabsorbed, undigested fats travel through the small and large intestines, ultimately causing the adverse effects associated with orlistat.

Medical Model for “Underwear Issues”

The medical model for the Underwear Issues of flatus with discharge and oily spotting that negatively impact orlistat product acceptance is anal incontinence (fecal incontinence) not pancreatic insufficiency or steatorrhea. Though the entire GI tract is comprised of involuntary smooth muscle, there are two areas (upper throat and anal muscles) that also possess voluntary muscle. The mouth and proximal one-third of the esophagus act to allow voluntary swallowing, while the anal apparatus allows for the voluntary release of flatus and defecation. The terminuses of the GI tract are under both voluntary control via striated muscles and involuntary reflex actions on these striated muscles.

There are two sphincters that control the distal terminus of the GI tract: the internal anal sphincter and the external anal sphincter. The internal anal sphincter is a physiologic sphincter where the terminal sigmoid colon penetrates through a window in the pelvic diaphragm. The pelvic diaphragm is composed of voluntarily controlled striated muscles. By tightening these levator muscles the window is closed via contraction (Kegle exercises). The external anal sphincter is a circular voluntarily controlled striated muscle that acts to maintain closure of the distal terminus of the GI tract. Muscle tone of both sphincters is under autonomic control via the vagus nerve, usually with the external sphincter completely closed and the internal sphincter relaxed. To insure that the sacrospinus nerves are intact, a simple clinical test is employed. The bulbocavernosa reflex is a reflex contracture of external anal sphincter muscle stimulated by the pinching of the clitoris or the glans penis. This reflex arc is involuntary and mediated by the distal spinal cord, just as is the patellar reflex (‘knee-jerk’ reflex).

The sigmoid colon is filled with sensory proprioceptors that sense pressure. These proprioceptors can usually differentiate the difference between pressure caused by feces in the sigmoid colon and gas in the sigmoid colon. Gas is allowed to escape by the simple voluntary relaxation of the external anal sphincter. Defecation is accomplished by relaxation of the external anal sphincter and increasing intra-abdominal pressure with the abdominal muscles, a voluntary action. The external anal sphincter and the internal anal sphincter are voluntarily contracted when the social situation is inappropriate for the discharge of flatus or feces.

Gas is produced by normal bacteria that aids in digestion. While this occurs to a small extent in the bowel, it predominantly occurs in the colon, and not at all in the stomach. The gas is propelled along the length of the GI tract by being intermingled with chyme and non-digested, ingested products (like bran) and the normal peristalsis of the smooth muscle. After any abdominal surgery, the bowel peristalsis is paralyzed and the gas accumulates in the bowel and colon. The ability of a post-operative patient to ‘pass gas’ signals the return of normal peristalsis and bowel function to the surgeon.

Flatus with Discharge (Voluntary Action)

Distension of the terminal sigmoid colon with gas is normal. This distension is perceived by the sensory proprioceptors. Voluntary relaxation of the external anal sphincter allows the gas to escape and relieves the distending pressures on the sigmoid sensory proprioceptors. However, large fat globules such as those resulting from the use of orlistat or other lipase inhibitor also can stimulate the sensory proprioceptors. It is this comparable sensory stimulation that can be interpreted as the need to ‘pass gas.’ Voluntary release of the gas will also discharge the large fat globules. This is best described as “playing paintball”—a pressurized propellant expelling a semi-solid projectile. To prevent this one can either prevent any large fat globules from presenting to the sigmoid colon (via a low fat diet), or ensure that fat presented to the sigmoid colon is in small droplets or absorbed by activated charcoal (as further described herein). This allows the use of orlistat on a moderate fat diet without the flatus with discharge, because the undigested fat is passed with the stools.

Oily Spotting (Involuntary Action)

Oily spotting is an involuntary reflex of the relaxation of the external anal sphincter in response to fat globule stimulation of the sigmoid sensory proprioceptors. Unlike the bulbocavernosa reflex that contracts the external anal sphincter, oily spotting is caused by the involuntary relaxation of the external anal sphincter, much like a gag reflex. Oily spotting usually occurs in the absence of gas, because if sigmoid distending gas were present, either the individual would visit the toilet, or experience the voluntary flatus with discharge. Once again, to prevent oily spotting, one needs to prevent large fat globules from stimulating the sigmoid colon sensory proprioceptors. Flatus with discharge and oily spotting are unique to the use of orlistat, not steatorrhea, in individuals with a moderate or high fat ingestion.

Reducing or Eliminating Underwear Issues

Applicant has discovered that the “Underwear Issues” associated with the use of orlistat and other lipase inhibitors can be reduced or prevented by administering a surfactant and activated charcoal along with the lipase inhibitor. This provides a method for treating obesity or an obesity-related condition, as well as a method of preventing the adverse events (i.e., side effects) associated with the use of orlistat or other lipase inhibitor. The lipase inhibitor (e.g., orlistat or cetilistat), surfactant and activated charcoal may be administered together as a pharmaceutical composition (or medicament) in a unit dosage form (e.g., one or more tablets or capsules, or even as a liquid suspension). Alternatively, the surfactant and activated charcoal may be administered separately from the lipase inhibitor (e.g., one or more capsules or tablets containing the surfactant and activated charcoal). In one particular embodiment, the activated charcoal is provided in enteric-coated form.

As used herein, the term “unit dosage form” means a physically discrete unit which contains the specified components in amounts selected so that a fixed number of the units is suitable to achieve a desired therapeutic effect. For example, when the lipase inhibitor, surfactant and activated charcoal are provided as an admixture in capsule or tablet form, a unit dose (i.e., the dose to be taken with a meal) may comprise a one, two, or three (or more) capsules/tablets, each of which is of similar composition. For example, in one embodiment described further herein, the composition comprises orlistat, simethicone and activated charcoal in a unit dosage form of a single capsule to be taken with a meal (i.e., right before, during or right after eating a meal).

In one embodiment, a pharmaceutical composition comprising a lipase inhibitor (e.g., orlistat or cetilistat), a surfactant (e.g., simethicone), and activated charcoal is administered to a patient in order to treat obesity or an obesity-related condition. The pharmaceutical composition is configured such that the lipase inhibitor, surfactant and activated charcoal are all dispersed in the patient's stomach and admixed with the stomach's contents. About one hour after the meal and composition have been ingested, pancreatic lipase enzyme is secreted by the pancreas through the ampulla of Vater and into the duodenum. There, the lipase inhibitor will bind with and inactivate the pancreatic lipase, thus preventing the digestion and absorption of the ingested fat. In addition, the surfactant will ensure that ingested fats remain in a very small state as an emulsion, rather then coalescing into large fat globules (as would be the case without the presence of the surfactant in the stomach and small intestine). The surfactant also ensures that the ingested fats are in a small enough state to be adequately absorbed by the activated charcoal. Once the ingested fat is in a small enough physical state due to the action of the surfactant, the activated charcoal will absorb the undigested unabsorbed fat within the stomach and small intestine. None of the three active ingredients, namely activated charcoal, simethicone and orlistat, are systemically absorbed into the body. All three remain in the GI Tract and are eliminated with defecation.

While the embodiment described in the preceding paragraph has been proven effective in preventing Underwear Issues associated with lipase inhibitors, in vitro testing has shown that the effectiveness of the lipase inhibitor is decreased due to the fact that some of the lipase inhibitor is absorbed by the activated charcoal and is therefore not available for binding to pancreatic lipase. For example, when 60 mg of orlistat was administered with simethicone and 400 mg of porous activated charcoal, in vitro testing showed a 30% decrease in the effectiveness of the orlistat. However, applicant has discovered that the activated charcoal does not need to be released in the stomach in order to effectively absorb the undigested, emulsified fat and prevent the Underwear Issues. Rather, the pharmaceutical composition may be configured such that the lipase inhibitor and surfactant are released in the stomach, and the activated charcoal is not released until the small intestine. By doing so, the lipase inhibitor becomes irreversibly bound to the pancreatic lipase enzyme (about 25%) in the very first 10 centimeters of the small intestines (the duodenum), prior to release of the activated charcoal, thus ensuring that the activated charcoal will not interfere with the ability of the lipase inhibitor to inactivate pancreatic lipase. For this reason, the activated charcoal in some embodiments of the compositions and methods herein is enteric-coated in order to maintain the full efficacy of the lipase inhibitor. By enterically coating the porous activated charcoal, the activated charcoal does not dissolve in the stomach and inactivate the orlistat, but rather dissolves in the mid small intestines (or lower) after the orlistat has been irreversibly bound to the lipase enzyme. Therefore, by using an enteric coated porous activated charcoal, the small undigested fat, emulsified in the chyme by actions of the emulsifying agent, can be chelated by the released porous activated charcoal in the distal small bowel.

Enteric coatings are designed to remain intact in the stomach but dissolve and release the underlying active substance in the intestine. Enteric coatings are polymers that are substantially insoluble in the acidic environment of the stomach, but are soluble in intestinal fluids at various pH's. Typically, a polymer coating is applied to tablets or pellets, and this coating prevents the dissolution of the tablet or pellets in the stomach. Since the pH of the small intestine increases along its length (from about 3.0-4.0 in the duodenum, to about 6.5 or higher in the terminal ileum), the specific dissolution properties of the enteric coating, as well as its thickness may be used to provide an enteric coating that will dissolve at a specific pH, in a specific region of the small intestine. By appropriate selection of the type of enteric coating as well as its thickness, the enteric coated activated charcoal is released in the desired location within the small intestine.

Enteric film coatings are typically 30-50 microns in thickness. Examples of polymer film coatings which may be used for the enteric coating of activated charcoal include:
cellulose acetate phthalate
hydroxypropyl methylcellulose phthalate
methylacrylic acid co-polymer type C (USP/NF based)
methylacrylic acid co-polymer type A (USP/NF based)

By way of example, one commercially-available type of enteric coating are EUDRAGIT® polymers available from Evonik Degussa Corporation. The EUDRAGIT® coatings are copolymers derived from esters of acrylic and methacrylic acid, with the physicochemical properties of the coating determined by functional groups attached to the copolymer backbone. By way of further example, activated charcoal may be combined with one or more binders and/or other suitable excipients to form pellets or tablets, which are then coated with an enteric polymer such as EUDRAGIT® L 100 or EUDRAGIT® L 12,5, which generally dissolve at a pH above 6.0. Such an enteric coating will release the activated charcoal primarily in the lower third of the small intestine. Alternatively, one of these polymers may be used in combination with another EUDRAGIT® polymer (e.g., EUDRAGIT® S 100 or EUDRAGIT® S 12,5) in order to provide an enteric coating which dissolves at a pH above 6.5 so that the activated charcoal will be primarily released in the terminal ileum. Of course these particular enteric coatings are merely exemplary and one skilled in the art will recognize that there are other suitable materials that would provide a tailored release profile whereby the activated charcoal is primarily or predominantly released in a specified region of the small intestine.

The enteric coating of pharmaceutical compositions is typically employed in two situations: to prevent the very acidic stomach environment from destroying, and therefore decreasing the effectiveness of, the ingested medication; and to prevent the ingested medication from eroding or injuring the gastric mucosa. Enteric coatings are widely used in the pharmaceutical industry for these two indications. However, the use of an enteric coating to prevent drug-drug interactions of multiple concurrently dosed medications in a defined anatomical space, the stomach, is not standard in medicine or the pharmaceutical industry.

Not only does the enteric coating on the activated charcoal prevent the charcoal from absorbing the lipase inhibitor before it is bound to pancreatic lipase enzyme, it also prevents drug-drug interactions between the activated charcoal and the simethicone. In addition, the enteric coating will also prevent the activated charcoal from absorbing (or limiting the absorption of) other drugs, vitamins or other nutrients ingested by the patient. Likewise, since the activated charcoal is needed to absorb emulsified fats, the enteric coating also prevents the charcoal from becoming overloaded with other absorbed materials which would limit the effectiveness of the charcoal in absorbing the emulsified fats in the small intestine.

As mentioned previously, the location where the enteric coating is dissolved and the activated charcoal is released may be controlled by the thickness of the particular coating used. Such manufacturing techniques are well known to those skilled in the art. In one example, the enteric coating is configured such that the activated charcoal is primarily (>50%), or even predominantly (>75%) released in the distal small intestine (from about the mid point of the bowel down). In another example, the enteric coating is configured such that the activated charcoal is primarily, or even predominantly released in the lower third of the small intestine. And in yet another example, the enteric coating is configured such that the activated charcoal is primarily, or even predominantly released in the terminal ileum (the portion of the small intestine located just before the large intestine, or bowel) or in the region defined by the terminal ileum and ascending colon. By configuring the enteric coating to release the activated charcoal in the lower third of the small intestine, the terminal ileum or the terminal ileum and ascending colon, the activated charcoal will still have time to absorb the undigested fats before the fats reach the distal colon, while limiting the ability of the activated charcoal to absorb other materials before they can be absorbed by the patient (e.g., other drugs, vitamins, nutrients, etc.).

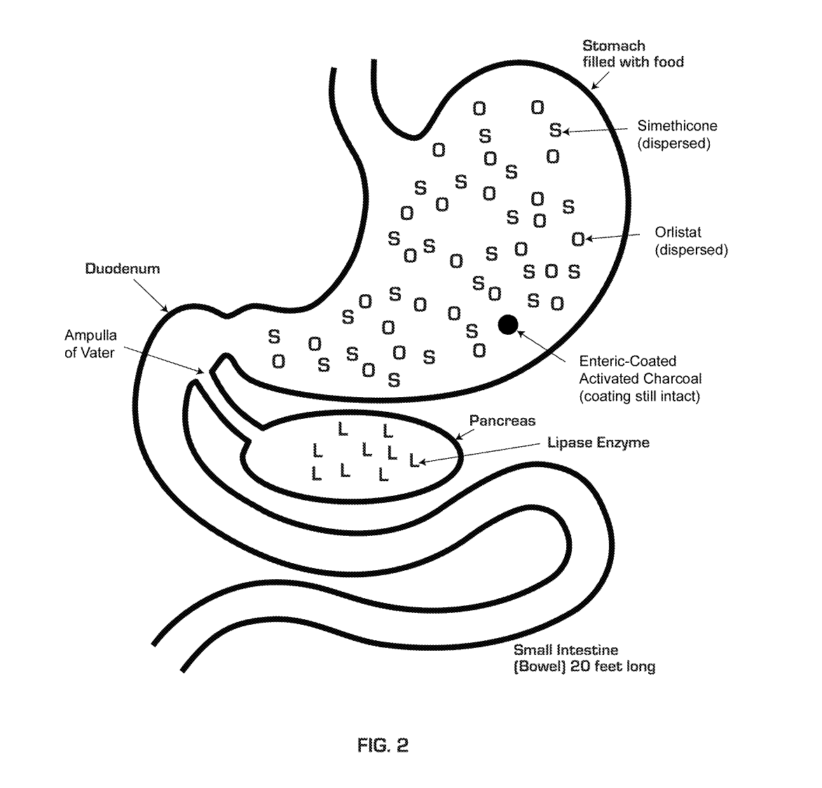

FIGS. 2-4 depict the manner in which the compositions and methods described herein promote weight loss while preventing the Underwear Issues associated with the use of lipase inhibitors such as orlistat and cetilistat. FIG. 2 is a diagrammatic representation of a portion of patient's GI tract about one-half of an hour after a meal has been consumed along with orlistat, simethicone and enteric-coated activated charcoal. The orlistat, simethicone and enteric-coated activated charcoal may have been ingested together as one or more capsules or tablets containing each of the three active components, or as one or more separate capsules or tablets of differing composition (e.g., one capsule containing orlistat and one capsule containing simethicone and enteric-coated activated charcoal, or individual capsules containing orlistat, simethicone and enteric-coated activated charcoal, respectively). As shown in FIG. 2, the stomach becomes distended in response to a meal, and the pancreas secretes pancreatic lipase enzyme into the pancreatic duct, through the ampulla of Vater and into the duodenum (the initial segment of the small intestine). As noted in FIG. 2, the orlistat and simethicone have been dispersed within the stomach, but the enteric-coated activated charcoal remains intact as one or more spheres (i.e., pellets or particles). The stomach rhythmically expels its contents into the duodenum where the food is admixed with the pancreatic lipase enzyme.

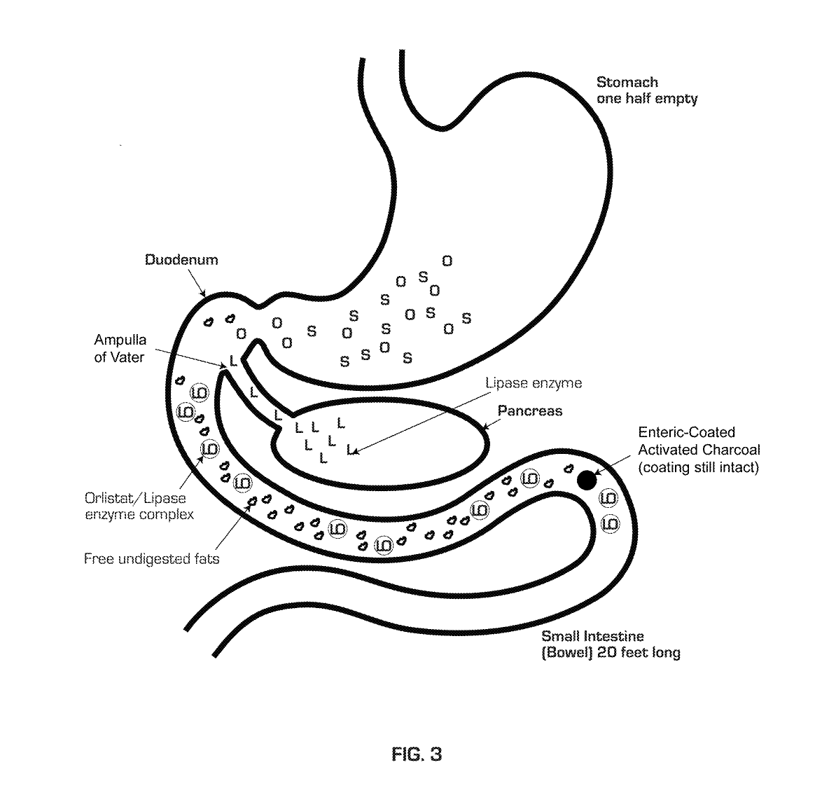

FIG. 3 depicts the stomach about one-half empty, at about one hour after consuming a meal. The pancreatic lipase enzyme has been and is being secreted from the pancreas through the ampulla of Vater. The orlistat is depicted binding with and inactivating the lipase enzyme in the duodenum, forming an irreversible bond therewith, with all of the orlistat being consumed. In addition, the emulsifying agent simethicone ensures that ingested fats remain in a very small state as an emulsion, rather then coalescing into large fat globules (as would be the case without the presence of the simethicone in the stomach and small intestine). The simethicone also ensures that the ingested fats are in a small enough state to be adequately absorbed by the activated charcoal. The enteric-coated activated charcoal remains intact in FIG. 3, as the coating has not yet dissolved in the small intestine.

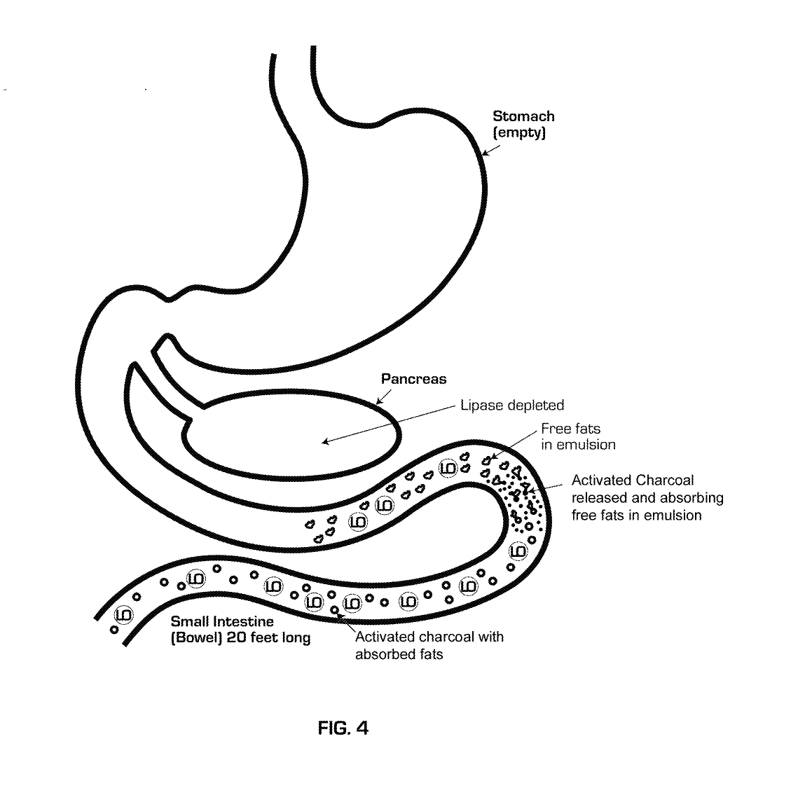

FIG. 4 depicts the stomach as empty, about two hours after consuming a meal. The pancreas is depleted of lipase enzyme, the free fats are still in an emulsion of small fat globules, and the enteric coating has dissolved to release the activated charcoal in the distal portion of the small intestine where the activated charcoal has begun to absorb the undigested fats. The activated charcoal continues to absorb the free fats in emulsion on their journey through and out the lower intestine. The orlistat is completely irreversibly bound to the lipase enzyme in the duodenum, and the dissolved activated charcoal primarily only absorbs the emulsified fats in order to prevent the “adverse events” of flatus with discharge and the involuntary rectal spotting.

Gastrointestinal transit is linear and progressive. As an analogy, it may be compared to two trains traveling from Boston to Miami. The 8AM breakfast train from Boston, is in NY city at noon, when the noon train leaves Boston bound for Miami. Since both trains use the same tracks (i.e. the GI Tract), and travel at the same speed, they will never collide. This is the linear and progressive normal bowel function that is the rationale for the use of the sequential actions of the lipase inhibitor and enteric-coated activated charcoal, which prevents the two from ever admixing, even though they are ingested concurrently.

With respect to the surfactant, simethicone may be employed. Alternatively, the surfactant may comprise one or more of: stearoxy dimethicone, dimethicone, methicone, bis-aminopropyl dimethicone, aminopropyl dimethicone, amodimethicone, amodimethicone hydroxystearate, behenoxy Dimethicone, C24-28 alkyl methicone, C30-45 alkyl methicone, simethicone, C30-45 alkyl dimethicone, cetearyl methicone and cetyl dimethicone. Simethicone may be used in liquid form (e.g., in a gelcap with one or more of the other active components) or as a solid (e.g., simethicone absorbed onto a granular carrier such as maltodextrin). The activated charcoal may comprise pharmaceutical grade, USP, activated charcoal, and may even be manufactured so as to maximize its ability to absorb undigested fats (e.g., high degree of porosity, size of pores, etc.). The lipase inhibitor may comprise, for example, a powder or pellets, such as described in U.S. Pat. No. 6,004,996.

As mentioned previously, the compositions (or medicaments) described herein may be formulated as tablets (including coated tablets, caplets, pills, etc.), capsules (including hard capsules, soft capsules, gelcaps, etc.), or even an emulsion or suspension (including not only aqueous suspensions, but also powdered or granulated mixtures which the patient or practitioner mixes with water or other liquid to form a suspension). The compositions will also include one or more pharmaceutically acceptable exipients, such as, but not limited to, fillers, binders, carriers, diluents, flavoring agents and sweeteners, colors, processing aids (e.g., glidants, granulating agents, lubricants, disintegrants etc.), and other materials known to those skilled in the art.

In one embodiment, the composition comprises a bilayer tablet, wherein one layer comprises orlistat (or cetilistat or other lipase inhibitor) in combination with simethicone. The other layer of the bilayer tablet comprises enteric-coated activated charcoal. The composition may be formulated such that a single bilayer tablet provides a unit dosage. Alternatively, the composition may be formulated such that two or more bilayer tablets provide a unit dosage.

As an alternative to a bilayer tablet, the composition may comprise a tablet having a core layer of enteric-coated activated charcoal, and one or more outer layers containing orlistat and simethicone (as a single layer or in separate layers).

In another embodiment, the composition is formulated as two or three distinct types of tablets. For example, one tablet may comprise orlistat (or cetilistat or other lipase inhibitor), and another tablet may comprise simethicone in combination with enteric-coated activated charcoal (e.g., as a bilayer tablet, with one layer containing the simethicone and the other layer containing the enteric-coated activated charcoal). Alternatively, the composition may be formulated as three distinct tablets—one containing the orlistat (or cetilistat or other lipase inhibitor), one containing the simethicone, and the third containing the enteric-coated activated charcoal.

In yet another embodiment, the composition comprises a capsule (hard capsule, soft capsule or gelcap) comprising orlistat (or cetilistat or other lipase inhibitor), simethicone and enteric-coated activated charcoal. The composition may be formulated such that a single capsule provides a unit dosage. Alternatively, the composition may be formulated such that two or more capsules provide a unit dosage.

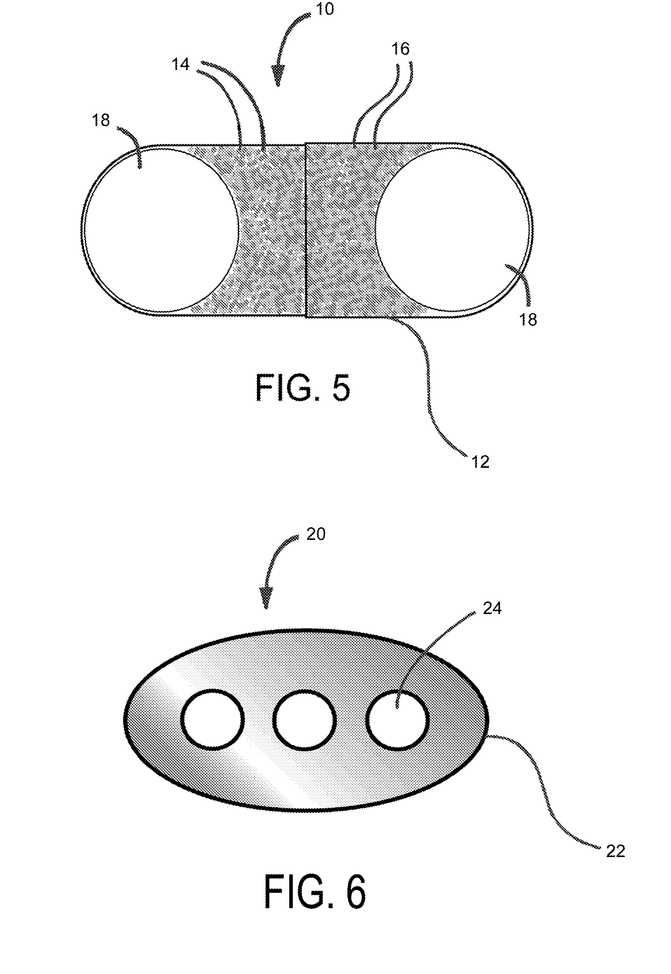

By way of further example, FIG. 5 depicts an exemplary unit dose medicament (10) for use in treating obesity or an obesity-related condition (e.g., metabolic syndrome, type II diabetes, hyperlipidemia, hypertension, dyslipidemia, etc.). Medicament (10) comprises a capsule (12) containing an admixture of orlistat pellets (or granules) (14) and granular simethicone (16) (also referred to as powdered simethicone). The orlistat pellets may be prepared, for example, in accordance with U.S. Pat. No. 6,004,996. If desired, a nonenteric coating (i.e., a coating that dissolves in the stomach) may be provided on the orlistat pellets and/or the granular simethicone in order to prevent the orlistat and simethicone from coming in contact within capsule (12) A pair of spherical enteric-coated activated charcoal pellets (18) are also provided in capsule (12). Of course other shapes and sizes of pellets may be used instead, or even enteric-coated tablets or capsules of enteric-coated activated charcoal which are sized to fit into capsule (12). In addition, any number and size of such enteric-coated tablets or pellets may be provided in the capsule in order to provide the appropriate dosage. Capsule (12) is configured to dissolve in the stomach so as to release the orlistat and simethicone in the patient's stomach. While the enteric-coated activated charcoal pellets or tablets are also released from capsule (12) in the stomach, the enteric-coating is configured so that the pellets or tablets remain intact until they reach the lower third of the small intestine. There, the enteric coating fully dissolves so as to release the activated charcoal after the orlistat has been irreversibly bound to pancreatic lipase enzyme. Although not shown in FIG. 5, medicament (10) further includes pharmaceutically-acceptable exipients, such as those previously described herein or others known to those skilled in the art. It should also be understood that a unit dose may comprise two capsules configured as shown in FIG. 5 in order to provide allow for smaller capsule sizes.

In one example of the compositions and methods described herein, a unit dose (i.e., the dose taken with a meal) comprises 30 mg to 240 mg of orlistat. In another example, a unit dose comprises 60 mg to 120 mg of orlistat. In yet another example, a unit dose comprises 120 mg of orlistat.

In another example of the compositions and methods described herein, a unit dose comprises 30 mg to 300 mg of simethicone (or other surfactant or combination of surfactants), and at least 200 mg of enteric-coated activated charcoal. In an alternative example, a unit does comprises 50 mg to 200 mg of simethicone, and 200 mg to 600 mg of enteric-coated activated charcoal.

In another exemplary composition, a unit dose comprises 30 mg to 240 mg of orlistat, 100 mg to 200 mg of simethicone, and 200 mg to 600 mg of enteric-coated activated charcoal.

### EXAMPLE 1

A capsule (as shown in FIG. 5) which, when orally ingested, dissolves in the stomach is filled with the following ingredients:

| | |
|---|---|
| Orlistat, pelleted | 120 mg |
| Simethicone, granular | 130 mg |
| Enteric-coated activated charcoal | 440 mg |
| Excipients | as required |

The enteric-coated activated charcoal is provided in the form of two enteric-coated round pellets housed within the capsule. The enteric coating is configured such that, when the capsules are ingested with a meal, the activated charcoal is primarily released in the lower third of the small intestine. It should also be pointed out that the above quantities of the three active ingredients in this example (as well as the other examples herein) refer to the actual amount of active ingredient in the capsule. For example, the 130 mg of granular simethicone refers to the weight of simethicone only, and does not include the weight of the carrier and other exipients used in forming the simethicone granules.

### EXAMPLE 2

A capsule (as shown in FIG. 5) which, when orally ingested, dissolves in the stomach is filled with the following ingredients:

| | |
|---|---|
| Orlistat, pelleted | 60 mg |
| Simethicone, granular | 125 mg |
| Enteric-coated activated charcoal | 300 mg |
| Excipients | as required |

The enteric-coated activated charcoal is provided in the form of two enteric-coated round pellets housed within the capsule, wherein the enteric coating is configured such that, when the capsules are ingested with a meal, the activated charcoal is primarily released in the distal portion of the small intestine.

### EXAMPLE 3

Two capsules (as shown in FIG. 5) which, when orally ingested, dissolve in the stomach are each filled with the following ingredients:

| | |
|---|---|
| Orlistat, pelleted | 60 mg |
| Simethicone, granular | 65 mg |
| Enteric-coated activated charcoal | 220 mg |
| Excipients | as required |

The enteric-coated activated charcoal is provided in the form of two enteric-coated tablets housed within the capsule, wherein the enteric coating is configured such that, when the capsules are ingested with a meal, the activated charcoal is primarily released in the terminal ileum.

The medicaments described herein may be packaged in any of a variety of ways. For example, unit dose packaging may be employed, such as a blister pack in the form of a strip or sheet of individually housed medicaments. As is well known to those skilled in the art, the web and lidding of the blister pack sheet or strip may even be scored such that individually housed unit doses of the medicament may be separated from the sheet or strip.

FIG. 6 depicts a top plan view of an alternative embodiment of a three-dose blister pack (20) comprising a web (22) having three pockets (24) formed therein. Each pocket (24) is sized and configured to receive and house a single unit dosage of a medicament described herein (e.g., in the form of one or more capsules or bilayer tablets). The lidding (or top web) which seals the pockets (24) is affixed to the underside of web (22), and is therefore not shown in FIG. 6. The three-dose blister pack (20) of medicaments provides one day's supply of medicament—with one of the unit dosages taken with each of three meals. The three-dose blister pack may be packaged in a container (e.g., a box or carton) containing 30 or 31 such blister packs (i.e., one month's supply of medicaments) along with printed instructions for use. Of course the three-dose blister pack shown in FIG. 6 is merely exemplary, as other forms of packaging may be used, such as other types of three-dose packages and other types of outer containers housing a plurality of three-dose packages.

While several compositions and methods have been discussed in detail above, it should be understood that the components, features, and methods of using the compositions discussed are not limited to the contexts provided above. Furthermore, additional and alternative suitable components, features, configurations, and methods of using the compositions, as well as various ways in which the teachings herein may be combined and interchanged, will be apparent to those of ordinary skill in the art in view of the teachings herein.

Having shown and described various versions in the present disclosure, further adaptations of the compositions and methods described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-31. (canceled)

32. A method of treating obesity or an obesity-related condition, comprising the step of administering a therapeutically effective amount of a lipase inhibitor in combination with an effective amount of a surfactant and an effective amount of enteric-coated activated charcoal.

33. The method of claim 32, wherein said lipase inhibitor is orlistat or cetilistat, and said surfactant is simethicone.

34. The method of claim 33, wherein said lipase inhibitor is administered separately from said simethicone and enteric-coated charcoal.

35. The method of claim 32 wherein said lipase inhibitor comprises orlistat, and said lipase inhibitor, simethicone and enteric-coated activated charcoal are administered together in a unit dosage form.

36. The method of claim 35, wherein said enteric coating is configured such that the activated charcoal is primarily released in the lower half of the small intestine.

37. The method of claim 35, wherein said enteric coating is configured such that the activated charcoal is primarily released in the lower third of the small intestine.

38. The method of claim 35, wherein said enteric coating is configured such that the activated charcoal is primarily released in the terminal ileum and ascending colon.

39. The method of claim 35, wherein said unit dosage form comprises from 30 to 240 mg of orlistat.

40. The method of claim 39, wherein said unit dosage form comprises from 60 to 120 mg of Orlistat.

41. The method of claim 39, wherein said unit dosage form comprises 30 to 300 mg of simethicone, and from 200 to 600 mg of enteric-coated activated charcoal.

42. The method of claim 35, wherein said unit dosage form comprises from 60 to 120 mg of orlistat, from 100 to 200 mg of simethicone, and from 200 to 600 mg of enteric-coated activated charcoal.

43. A pharmaceutical composition in unit dosage form, comprising a therapeutically effective amount of a lipase inhibitor in combination with an effective amount of a surfactant, and an effective amount of enteric-coated activated charcoal.

44. The pharmaceutical composition of claim 43, wherein said lipase inhibitor comprises orlistat and said surfactant comprises simethicone, and wherein said unit dosage form comprises from 30 to 240 mg of orlistat.

45. The pharmaceutical composition of claim 44, wherein said unit dosage form comprises from 60 to 120 mg of orlistat.

46. The pharmaceutical composition of claim 45, wherein said unit dosage form comprises 30 to 300 mg of simethicone.

47. The pharmaceutical composition of claim 44, wherein said unit dosage form comprises from 60 to 120 mg of orlistat, from 100 to 200 mg of simethicone, and from 200 to 600 mg of enteric-coated activated charcoal.

48. The pharmaceutical composition of claim 47, wherein said unit dosage form comprises at least one orally-ingestible tablet or capsule.

49. The pharmaceutical composition of claim 48, wherein said unit dosage form comprises at least one orally-ingestible capsule, wherein said enteric-coated activated charcoal comprises a plurality of enteric-coated pellets contained with said at least one capsule, and further wherein said capsule and said enteric coating are configured such that said orlistat and said simethicone are primarily released in the stomach, and said activated charcoal is primarily released in the lower third of the small intestine.

50. The pharmaceutical composition of claim 49, wherein said unit dosage form comprises 120 mg of orlistat, 130 mg of simethicone and 440 mg of activated charcoal.

51. A package containing three separately housed unit doses of the pharmaceutical composition of claim 44.
